(19) United States
(12) Patent Application Publication
(10) Pub. No.: US 2024/0156996 A1
(43) Pub. Date: May 16, 2024

(54) BIOCOMPATIBLE RADIONUCLIDE-CONTAINING COMPOSITIONS AND METHODS OF USE

(71) Applicant: UT-Battelle, LLC, Oak Ridge, TN (US)

(72) Inventors: Sandra M. Davern, Knoxville, TN (US); Miguel Toro Gonzalez, Knoxville, TN (US); Ilja Popovs, Knoxville, TN (US); Santa Jansone-Popova, Knoxville, TN (US)

(21) Appl. No.: 18/385,428

(22) Filed: Oct. 31, 2023

Related U.S. Application Data
(60) Provisional application No. 63/421,290, filed on Nov. 1, 2022.

(51) Int. Cl. A61K 51/04; A61K 9/51; A61P 35/00; B82Y 5/00

(52) U.S. Cl. CPC A61K 51/0455; A61K 9/5146; A61P 35/00; A61K 2121/00; A61K 2123/00; B82Y 5/00

(57) ABSTRACT

A biocompatible radionuclide-containing composition comprising: (i) a radionuclide-containing core, which includes complexed or uncomplexed radionuclide atoms; and (ii) a carrier encapsulating the radionuclide-containing core, wherein the carrier may be attached to a targeting moiety that can selectively transport the composition to a specific cell type when introduced into an organism, or the carrier may be attached to a moiety that evades an immune system of an organism. Also described is a method of treating cancer in a subject by administering to the subject a pharmaceutically effective amount of the composition described above. Also described is a method of treating an infection in a subject by administering to the subject a pharmaceutically effective amount of the composition described above. Also described is a method of imaging biological tissue in a subject by administering the composition to the subject and imaging biological tissue in the subject by a nuclear medicine imaging technique.

### CROSS REFERENCE TO RELATED APPLICATION

The present application claims benefit of U.S. Provisional Application No. 63/421,290, filed on Nov. 1, 2022, all of the contents of which are incorporated herein by reference.

### STATEMENT REGARDING FEDERALLY SPONSORED RESEARCH

This invention was made with government support under Prime Contract No. DE-AC05-000R22725 awarded by the U.S. Department of Energy. The government has certain rights in the invention.

### FIELD OF THE INVENTION

The present invention generally relates to biocompatible radionuclide compositions useful in radiopharmaceutical therapy. The present invention more particularly relates to methods of treatment and imaging of biological tissue using such compositions, particularly chelated and nanoparticle compositions containing one or more therapeutic radionuclides. The present invention further relates to methods for selective targeting of biocompatible radionuclide compositions to specific biological tissue, such as tumor cells.

### BACKGROUND

Alpha, beta, and Auger emitting radionuclides have long been used for treating cancer. However, a longstanding problem has been the difficulty in selectively directing the therapeutic radionuclides to desired cellular tissue, such as cancerous tissue. Selective targeting of biological tissue is important for maximizing of the therapeutic effect of the treatment and minimizing the side effects associated with off targeting. Although there are ongoing efforts to improve the degree of selective targeting, the degree of selectivity remains largely deficient. Moreover, much of the conventional art relies on inorganic nanoparticles, which are generally not biocompatible.

### SUMMARY

In one aspect, the present disclosure is directed to a biocompatible radionuclide-composition that includes the following components: (i) a radionuclide-containing core; and (ii) a carrier encapsulating the radionuclide-containing core. In some embodiments, the carrier is attached to a targeting moiety capable of selectively transporting the composition to a specific cell type when introduced into an organism. The composition is typically in the form of particles, such as nanoparticles. By virtue of their unique construction, the particles advantageously trap and retain therapeutic radionuclides and their byproducts at the cancer site. This ability is important to maximize the therapeutic effect of the treatment and minimize associated side effects. These biocompatible particles can also be tagged with molecules that will allow them to specifically seek out cancer cells anywhere in the body. This technology has the potential to be combined with precision chemotherapy and immunotherapy for more accurate treatment of local and metastatic disease while minimizing side effects to patients.

In particular embodiments, the present disclosure has demonstrated the use of biocompatible polylactic-co-glycolic acid (PLGA) nanoparticles as delivery vehicles for the alpha emitter Ac-225. Encapsulation of this radionuclide was achieved by first chelating it with a lipophilic version of the BLph en chelator. The ability of these Ac-225-PLGA nanoparticles to kill cancer cells has also been demonstrated. PLGA nanoconstructs loaded with chelated radionuclides (alpha, beta and Auger emitters) are herein demonstrated to be effective at killing cancer cells.

In another aspect, the present disclosure is directed to a method of treating cancer in a subject, the method comprising administering to the subject a pharmaceutically effective amount of a biocompatible radionuclide-containing composition described above. The cancer may be, for example, prostate cancer, ovarian cancer, breast cancer, leukemia, colorectal cancer, lymphoma, or neuroblastoma. In some embodiments, the carrier of the composition is attached to a targeting moiety capable of selectively transporting the composition to cancer cells when introduced into an organism.

In another aspect, the present disclosure is directed to a method of treating an infectious disease in a subject, the method comprising administering to the subject a pharmaceutically effective amount of a biocompatible radionuclide-containing composition described above. The disease may be, for example, a bacterial, viral, or fungal infection. In some embodiments, the infectious disease is a drug-resistant infectious disease. In some embodiments, the method is used to treat a joint that is infected or at risk of becoming infected. In some embodiments, the carrier of the composition is attached to a targeting moiety capable of selectively transporting the composition to infected cells or joint cells when introduced into an organism.

In yet another aspect, the present disclosure is directed to a method of imaging biological tissue in a subject, the method comprising administering to the subject a pharmaceutically effective amount of a biocompatible radionuclide-containing composition described above. The imaging is typically made possible by a nuclear medicine imaging technique.

### BRIEF DESCRIPTION OF THE DRAWINGS

FIG. 1 is a schematic showing a double-emulsion solvent evaporation method for producing a PLGA-chelator radioisotope complex.

FIG. 2 is a plot showing the favorable encapsulation of ²²⁵Ac within PLGA nanoparticles in a hydrophobic environment.

FIG. 3 is a plot showing the enhanced retention of ²²⁵Ac within PLGA nanoparticles when encapsulated as ²²⁵Ac-BLPhen complex. Release of ²²⁵Ac after encapsulation of free ²²⁵Ac in a methanol/deionized water solution or in ammonium acetate are shown as a reference.

FIG. 4 is a plot showing the increased retention of ²²¹Fr, first decay daughter of ²²⁵Ac, within PLGA nanoparticles after encapsulation of as ²²⁵Ac-BLPhen complex. Release of ²²¹Fr after encapsulation of free ²²⁵Ac in a methanol/deionized water solution or in ammonium acetate are shown as a reference.

FIGS. 5A-5F are bar plots showing the highly cytotoxic effect of PLGA nanoparticles encapsulating ²²⁵Ac-BLPhen complex on murine E0771 (FIGS. 5A and 5B), human MCF7 (FIGS. 5C and 5D), and MDA-MB-231 (FIGS. 5E and 5F). Free ²²⁵Ac and ²²⁵Ac-BLPhen complex were used as controls. Left-side plots (FIGS. 5A, 5C, and 5E) correspond to viability assessed 1 h post exposure to ²²⁵Ac, whereas right-side plots (FIGS. 5B, 5D, and 5F) correspond to viability assessed 72 h post exposure.

FIGS. 6A-6C are graphs showing the cytotoxic effect of PLGA nanoparticles encapsulating ²²⁵Ac-BLPhen complex based on particle concentration. Viability of murine E0771 (FIG. 6A), human MCF7 (FIG. 6B), and MDA-MB-231 (FIG. 6C) was assessed 1 h post exposure to ²²⁵Ac.

### DETAILED DESCRIPTION

The term “alkyl,” as used herein, refers to a linear or branched alkyl hydrocarbon group containing from, for example, about 1 to about 12 carbon atoms, preferably from about 1 to about 8 carbon atoms, more preferably from about 1 to about 6 carbon atoms. Examples of such substituents include methyl, ethyl, n-propyl, isopropyl, n-butyl, sec-butyl, isobutyl, tert-butyl, pentyl, isoamyl, hexyl, octyl, decyl, dodecyl, and the like.

The term “aryl,” as used herein, refers to an unsubstituted or substituted aromatic carbocyclic substituent, as commonly understood in the art, and includes monocyclic and polycyclic aromatics such as, for example, phenyl, biphenyl, tolyl, anisolyl, naphthyl, anthracenyl and the like. An aryl substituent generally contains from, for example, about 5 to about 30 carbon atoms, more typically about 6 to about 18 carbon atoms, more typically from about 6 to about 14 carbon atoms, and more typically from about 6 to about 10 carbon atoms.

The term “alkoxy” (OR), as used herein, generally refers to a linear or branched alkyl or other hydrocarbon group (R) that is attached to divalent oxygen. The alkyl group can be any of those described above. Examples of such substituents include methoxy, ethoxy, t-butoxy, and the like.

In one aspect, the present disclosure is directed to a biocompatible radionuclide-containing composition (i.e., delivery platform complex or “composition”) that includes the following components: (i) a radionuclide-containing core; and (ii) a carrier encapsulating the radionuclide-containing core. The composition is typically in the form of particles, such as nanoparticles. In embodiments, the core is approximately or precisely spherical in shape and has a diameter of up to or less than 1 micron. The diameter of the particle core (or the core encapsulated by the carrier) may be, for example, 5, 10, 20, 50, 100, 150, 200, 250, 300, 400, 500, 600, 700, 800, or 900 nm, or a size within a range bounded by any two of the foregoing values (e.g., 5-900 nm, 10-900 nm, 20-900 nm, 50-900 nm, 5-500 nm, 10-500 nm, 20-500 nm, 50-500 nm, 5-200 nm, 10-200 nm, 20-200 nm, 50-200 nm, 5-100 nm, 10-100 nm, 20-100 nm, 50-100 nm, 5-50 nm, 10-50 nm, 20-50 nm, 5-20 nm, 10-20 nm, or 5-10 nm).

The radionuclide-containing core (i.e., “core”) typically contains a large multiplicity of radionuclide atoms, such as at least or more than 20, 50, 100, 500, 1000, 5000, 10,000, 50,000, or 100,000 radionuclide atoms. The radionuclide atoms can be any of the radionuclides known in the medical arts to be useful for treating or imaging of a biological organism. The radionuclide atoms are typically selected from alpha emitting, beta emitting, or Auger emitting. Some examples of alpha emitting radionuclides include actinium-225, thorium-227, thorium-226, radium-223, radium-224, lead-212, astatine-211, uranium-230, bismuth-231, and combinations thereof. Some examples of beta emitting radionuclides include lutetium-177, samarium-153, yttrium-90, strontium-89, iodine-131, and combinations thereof. Some examples of Auger emitting radionuclides include indium-111, iodine-125, iodine-123, platinum-193m, platinum-195m, terbium-161, and combinations thereof. Some examples of imaging radionuclides include iodine-131, iodine-124, iodine-123, technetium-99m, thallium-201, indium-111, gallium-67, cerium-134, copper-64, and combinations thereof.

In some embodiments, the radionuclide-containing core contains a radionuclide-chelator complex. The radionuclide-chelator complex contains a radionuclide atom complexed (coordinated) with a chelator compound. In some embodiments, the radionuclide-chelator complex contains a multiplicity of radionuclide atoms interconnected with each other by linking chelator molecules to form an extended framework. The linking chelator molecules may be organic or inorganic molecules. The foregoing type of composition may be a metal-organic framework (MOF). Numerous chelator compounds are known to suitably chelate to numerous radionuclides to form stable complexes suitable for administration into an organism. The radionuclide-chelator complex can be any of those known in the art. Some well known types of chelator compounds include those based on phenanthroline, dipicolinate, or nitrogen-containing macrocycle compounds.

Some types of phenanthroline-based chelators include:

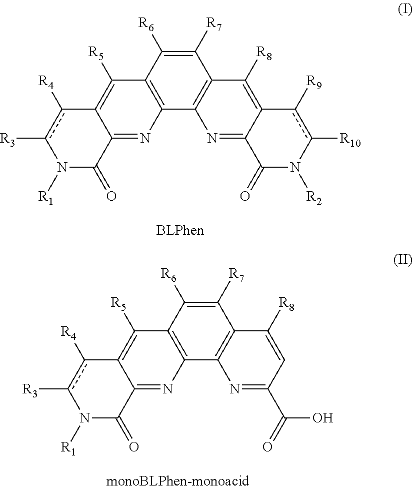

In Formulas (I) and (II), R₁, R₂, R₃, R₄, R₅, R₆, R₇, R₈, R₉, and R₁₀ are each independently selected from hydrogen atom, linear or branched alkyl or alkenyl groups (R) containing 1-12 carbon atoms, OH, alkoxy groups (OR), and cyclic groups (e.g., cycloalkyl, cycloalkenyl, aryl, heterocycloalkyl, and heteroaryl groups). The dashed lines indicate the presence of a single or double bond (i.e., an optional double bond). Any adjacent substituents among R₁-R₁₀ may or may not be permitted to interconnect to form one or more additional fused rings. In some embodiments, R₃ and R₄ may interconnect to form a ring and/or R₉ and R₁₀ may interconnect to form a ring. In other embodiments, R₃ and R₄ do not interconnect to form a ring and/or R₉ and R₁₀ do not interconnect to form a ring. In some embodiments, R₆ and R₇ may or not interconnect to form another fused ring.

Some particular examples of phenanthroline-based chelators include:

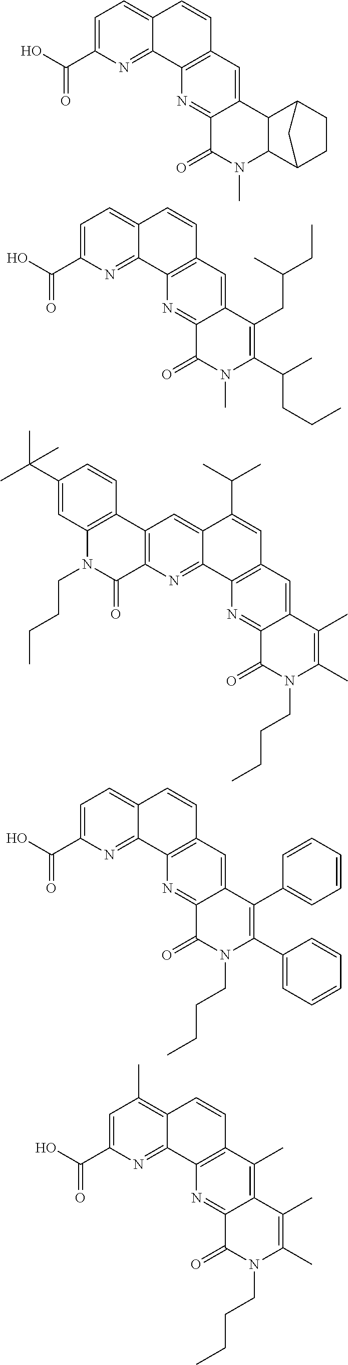

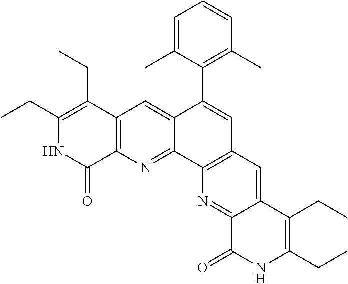 [SMILES: CCc1[nH]c(=O)c2nc3c(cc(-c4c(C)cccc4C)c4cc5c(CC)c(CC)[nH]c(=O)c5nc43)cc2c1CC]

In some embodiments, the phenanthroline-based chelator excludes chelator compounds in which any one or more of the following conditions are present: (i) R₁ and R₂ are both hexyl groups; (ii) R₅-R₈ are hydrogen atoms; (iii) R₃ and R₄ interconnect to form a ring, or more particularly, a 1,4-cyclopetanyl substituent; (iv) R₉ and R₁₀ interconnect to form a ring, or more particularly, a 1,4-cyclopetanyl substituent.

In some embodiments, the phenanthroline-based chelator excludes the following structure:

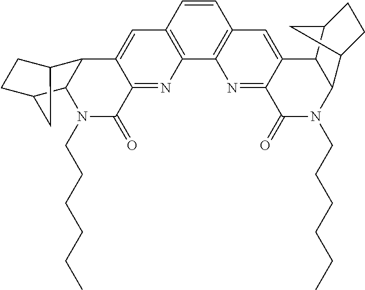 [SMILES: CCCCCCN1C(=O)c2nc3c(ccc4cc5c(nc43)C(=O)N(CCCCCC)C3C4CCC(C4)C53)cc2C2C3CCC(C3)C21]

In other embodiments, the foregoing structure can be included within the scope of Formula (I).

Some types of dipicolinate chelator compounds include:

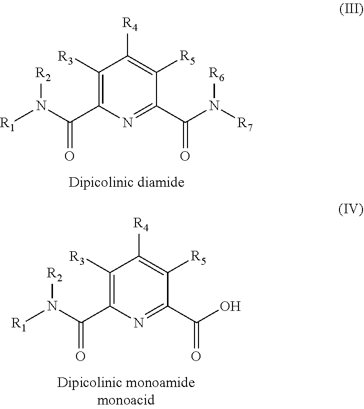

In Formulas (III) and (IV), R₁-R₇ are as defined earlier above. In each formula, R₁ and R₂ may or may not interconnect, R₂ and R₃ may or may not interconnect, R₃ and R₄ may or may not interconnect, R₄ and R₅ may or may not interconnect, R₅ and R₆ may or may not interconnect, and R₆ and R₇ may or may not interconnect.

Some particular examples of dipicolinate chelator compounds include:

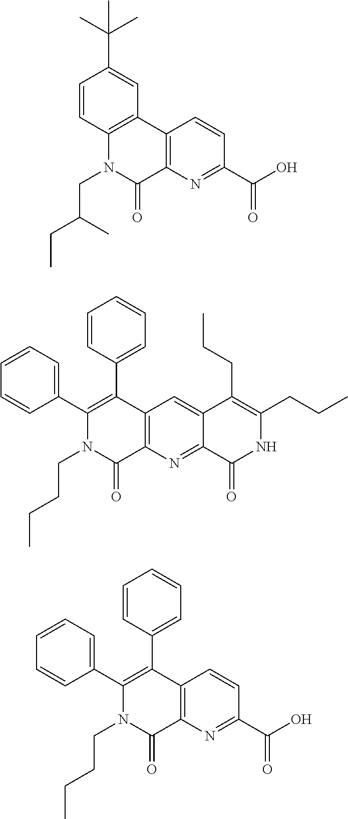 [SMILES: CCC(C)Cn1c(=O)c2nc(C(=O)O)ccc2c2cc(C(C)(C)C)ccc21 ; CCCCn1c(-c2ccccc2)c(-c2ccccc2)c2cc3c(CCC)c(CCC)[nH]c(=O)c3nc2c1=O ; CCCCn1c(-c2ccccc2)c(-c2ccccc2)c2ccc(C(=O)O)nc2c1=O]

Some types of nitrogen-containing macrocycle compounds include those within the following generic formula:

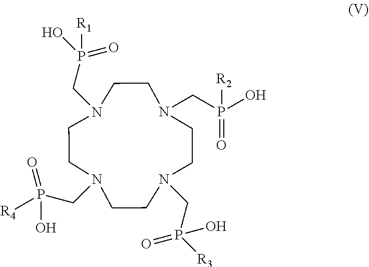

In Formula (V), R₁, R₂, R₃, and R₄ are independently as defined earlier above, and they may alternatively or in addition be independently selected from OH, OR, and NR₂ groups, wherein the R groups are independently selected and typically selected from linear and branched alkyl groups and may or may not be interconnected in the NR₂ group. In some embodiments, R₁, R₂, R₃, and R₄ of Formula (V) are independently selected from H, R, OR, and NR₂, wherein R is independently selected from hydrogen, alkyl, aryl, fluoroalkyl, oxyalkyl, aliphatic, aromatic, heteroaromatic, and derivatives of these structures.

Some particular examples of nitrogen-containing macrocycle compounds include:

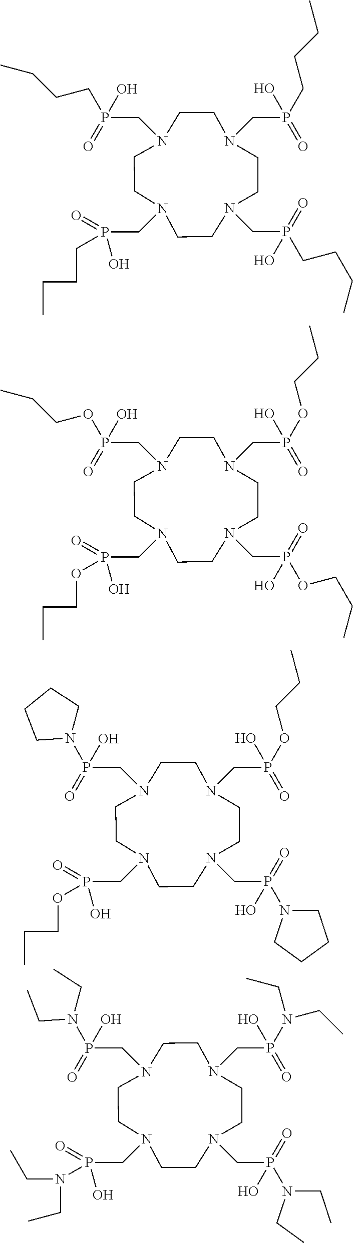

The nitrogen-containing macrocycle compound may, in some embodiments, contain carboxy groups instead of or in combination with phosphoester or phosphamide groups. Some examples of such chelating macrocycles include H₂macropa, DOTA (tetraxetan), DOTAGA, DOTMP, DEPA, TETA, PCTA, PEPA, HEHA, NOTA, and NODAGA. The chelator compound may alternatively or in addition be a common non-macrocyclic compound, such as EDTA, EDTMP, H₂CHXdodpa, H₄CHoctapa, H₄octapa, and H₅decapa.

To prepare a chelator compound radioisotope complex described herein, a chelator compound, such as any of those within Formulas (I)-(V), are complexed with an appropriate radioisotope. Methods of preparing radionuclide complexes are well known in the art. For example, the radioisotope can be added to water and treated with an equimolar amount of a chelator compound of any of formulas (I)-(V). The chelator compound can be added as an aqueous solution or suspension. Dilute acid or base can be added (where appropriate) to maintain a suitable pH. Heating at temperatures as high as 100° C. for periods of up to 24 hours or more may be employed to facilitate complexation, depending on the radioisotope, the chelator compound, and their concentrations.

The radionuclide-containing core may alternatively be an extended network (framework) of radionuclide atoms interconnected with non-chelating ligands and/or atoms (e.g., oxide or sulfide). Each non-chelating ligand can typically interconnect two or three radionuclide atoms. The non-chelating ligands may be organic or inorganic molecules. Some examples of non-chelating ligands include carboxylates (e.g., oxalate, terephthalate, or trimesic acid), polyols (e.g., ethylene glycol and phloroglucinol), and/or amines (e.g., pyrazine or bipyridine). In some embodiments, the core contains an extended network of radionuclide atoms interconnected with oxide or sulfide atoms and/or non-chelating ligands (e.g., acetate, formate, phenoxide, dialkylamines, and phosphonates). The extended framework may or may not also include radionuclide chelators.

The radionuclide-containing core may further include one or more solvents. In the case of two or more solvents, the two or more solvents may be immiscible, as in an oil-in-water or water-in-oil-in-water (W/O/W) emulsion. The radionuclide atoms may be in the form of a salt, a radionuclide-chelator complex, or extended network in the solvent, and may be dissolved or suspended within the solvent. The solvent is typically non-toxic, such as water or an aqueous solution.

The carrier encapsulates the radionuclide-containing core described above. The carrier can be any of the carriers well known in the medical arts for use in transporting a drug or biologically active material in an organism. In some embodiments, the carrier has a biocompatible polymeric composition. The biocompatible polymeric composition can be, for example, a polyester, polyamide (e.g., polyamino acid), poly(ester amide), polyurethane, polyurea, polycarbonate, polyalkylene oxide (e.g., polyethylene oxide or polypropylene oxide), or polysaccharide (e.g., chitosan), or mixtures or copolymers thereof. In other embodiments, the carrier is a micelle or liposome. The carrier may also be selected to be neutrally charged, positively charged, or negatively charged.

In particular embodiments, the polymeric composition is or includes a polyester. The polyester may be or include, for example, poly(lactic-co-glycolic acid) PLGA, poly(lactic-co-glycolic acid) copolymers PLGA-PEG or PLGA-PEG-PLGA, poly(lactic acid) (PLA), poly(glycolic acid) (PGA), poly(caprolactone) (PCL), poly(caprolactone) copolymers, poly(malic acid) (PMLA), poly(alkyl cyanoacrylates) (PACAs), poly(ortho esters), poly(phosphoesters), and poly(anhydrides), such as poly(sebacic acid), poly(adipic acid) and poly(terephthalic acid).

In some embodiments, the encapsulating carrier is attached (i.e., coupled or covalently bound) to a targeting moiety capable of selectively transporting the radionuclide-containing composition to a specific cell type when introduced into an organism. Examples of suitable targeting moieties include, but are not limited to, amino acids, peptides, oligopeptides, oligonucleotides, nucleic acids (e.g., aptamers), antibodies (e.g., monoclonal antibodies or antibody fragments), lectins, small molecules, and others. In some embodiments, the targeting moiety is specifically selected for binding to cancer cells or specific cancer cells. Some targeting moieties specifically suited for targeting cancer cells include folate (or folic acid), transferrin, aptamers, antibodies that target antigens specific to cancer cells (e.g., anti-EGFR antibodies or humanized A33 monoclonal antibodies), oligopeptides (e.g., RGD sequence and Angiopep-2), prostate-specific membrane antigen (PSMA) targeting probes (including anti-PSMA antibodies, and also small molecules containing a Glu-NH—CO—NH-Lys core), and proteins (e.g., inhibitors of matrix metalloproteinases). In other embodiments, the targeting moiety is specifically selected for binding to collagen, intra-articular cartilage, or chondrocytes. The targeting moiety may be, for example, an oligopeptide, such as WYRGRL, or a protein (e.g., antibodies to collagen type II). In other embodiments, the targeting moiety is specifically selected for binding to an infectious organism, such as a bacterium, virus, fungus, or parasite, or a biofilm of any of these. The targeting moiety may be, for example, chitosan, poly(L-histidine), trialkylammonium, or other positively charged moiety; a carbohydrate or glycan that targets an infectious organism; or a targeted delivery vector.

In other embodiments, the carrier is attached to a moiety that evades an immune system of an organism. Examples of suitable moieties for evading an immune system include, but are not limited to, PEGylation, sialic acid, lectins, and carbohydrate moieties.

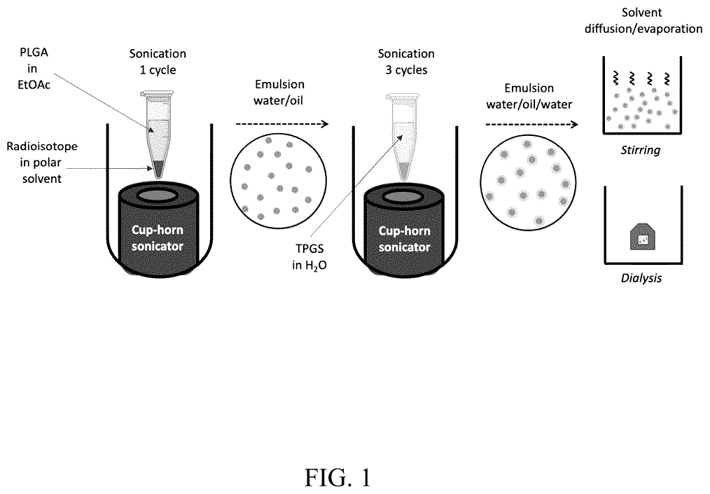

The biocompatible radionuclide-containing compositions described above can be synthesized by methods well known in the art. For example, as shown in the Examples herein, the compositions can be formed using a double-emulsion solvent evaporation method in which a multiplicity of radionuclide atoms (which may or not be chelated) are encapsulated within a biocompatible carrier, such as a polymeric carrier. An exemplary double-emulsion solvent evaporation method is shown schematically in FIG. 1. The compositions can alternatively be produced by, for example, single emulsion solvent evaporation or nanoprecipitation. These and other methods are described in, for example, Zielenska et al., Molecules, 25(16), 3731, 2020, all of which is herein applicable and incorporated by reference.

In another aspect, the invention is directed to pharmaceutical compositions that contain any of the above-described biocompatible radionuclide-containing compositions dispersed in a pharmaceutically acceptable vehicle (excipient). The composition is dispersed in the pharmaceutically acceptable vehicle by either being mixed (e.g., in solid form with a solid carrier) or dissolved or emulsified in a liquid excipient. The pharmaceutical composition may or may not also be formulated together with one or more additional active ingredients or adjuvants that improve the overall efficacy of the pharmaceutical composition, particularly as relates to the treatment of cancer, infection by a microbe (e.g., a bacterium, virus, or fungus), or imaging of biological tissue.

The composition and vehicle (liquid or solid) may be formulated into pharmaceutical compositions and dosage forms according to methods well known in the art. The pharmaceutical compositions of the present invention may be specially formulated for administration in liquid or solid form. In some embodiments, the pharmaceutical formulation is formulated for oral administration (e.g., as tablets, capsules, powders, granules, pastes, solutions, suspensions, drenches, or syrups); parenteral administration (e.g., by subcutaneous, intramuscular or intravenous injection as provided by, for example, a sterile solution or suspension); topical application (e.g., as a cream, ointment, or spray); intravaginal or intrarectal administration (e.g., as a pessary, cream or foam); sublingual or buccal administration; ocular administration; transdermal administration; or nasal administration. In some embodiments, the pharmaceutical composition is a liquid formulation designed for administration by injection.

The pharmaceutical composition can be made suitable for use as a medicament for human or animal use. The pharmaceutical composition may be, for example, an injectable formulation, a liquid, cream or lotion for topical application, an aerosol, a powder, granules, tablets, suppositories, or capsules, such as, for example, enteric coated capsules, or the like. The pharmaceutical composition may also be delivered as part of a lipid formulation, such as, for example, an emulsion or a liposome preparation. The pharmaceutical compositions are preferably sterile, non-pyrogenic and isotonic preparations, optionally with one or more of the pharmaceutically acceptable additives listed below. Pharmaceutical compositions are preferably stable compositions which may contain one or more of the following: a stabilizer, a surfactant, preferably a nonionic surfactant, and optionally a salt and/or a buffering agent. The pharmaceutical composition may be in the form of an aqueous solution, or may be in a lyophilized form. The stabilizer may, for example, be an amino acid, such as for instance, glycine; or an oligosaccharide, such as for example, sucrose, tetralose, lactose, or a dextran. Alternatively, the stabilizer may be a sugar alcohol, such as for instance, mannitol; or a combination thereof. Preferably, the stabilizer or combination of stabilizers constitutes from about 0.1% to about 10% weight for weight of the composition. The surfactant may be a nonionic surfactant, such as a polysorbate. Some examples of suitable surfactants include Tween20, Tween80; a polyethylene glycol or a polyoxyethylene polyoxypropylene glycol, such as Pluronic F-68 at from about 0.001% (w/v) to about 10% (w/v). The salt or buffering agent may be any salt or buffering agent, such as for example, sodium chloride, or sodium/potassium phosphate, respectively. Preferably, the buffering agent maintains the pH of the pharmaceutical composition in the range of about 5.5 to about 7.5. The salt and/or buffering agent is also useful to maintain the osmolality at a level suitable for administration to a human or an animal. Preferably, the salt or buffering agent is present at a roughly isotonic concentration of about 150 mM to about 300 mM.

The pharmaceutical composition may additionally contain one or more conventional additives. Some examples of such additives include a solubilizer, such as, for example, glycerol; an antioxidant, such as, for example, benzalkonium chloride (a mixture of quaternary ammonium compounds, known as “quats”), benzyl alcohol, chloretone or chlorobutanol; an anesthetic agent, such as, for example, a morphine derivative; or an isotonic agent, such as described above. As a further precaution against oxidation or other spoilage, the pharmaceutical compositions may be stored under nitrogen or argon gas in vials sealed with impermeable stoppers.

The phrase “pharmaceutically acceptable” refers herein to those compounds, materials, compositions (e.g., acids or bases), and/or dosage forms which are, within the scope of sound medical judgment, suitable for administration to a subject. The phrase “pharmaceutically acceptable vehicle,” as used herein, refers to a pharmaceutically acceptable liquid or solid filler, diluent, carrier, manufacturing aid (e.g., lubricant, talc magnesium, calcium or zinc stearate, or stearic acid), solvent, or encapsulating material, that serves to carry the therapeutic composition for administration to the subject. Each vehicle (excipient or diluent) should be “acceptable” in the sense of being compatible with the other ingredients of the formulation and physiologically safe to the subject. Any of the vehicles/excipients known in the art can be suitable herein depending on the mode of administration.

Some examples of materials that can serve as pharmaceutically acceptable vehicles include water; aqueous and non-aqueous solutions; isotonic saline; pH buffering agents; anti-oxidants; bacteriostats; sugars (e.g., lactose, glucose, sucrose, and oligosaccharides, such as sucrose, trehalose, lactose, or dextran); solutes that render the formulation isotonic with the blood of the intended recipient; and aqueous and non-aqueous sterile suspensions that can include suspending agents, solubilizers, thickening agents, stabilizers, and preservatives. Other excipients, more typically used in solid dosage forms, may also be included, e.g., starches (e.g., corn and potato starch); cellulose and its derivatives (e.g., sodium carboxymethyl cellulose, ethyl cellulose and cellulose acetate); gelatin; talc; waxes; oils (e.g., peanut oil, cottonseed oil, safflower oil, sesame oil, olive oil, corn oil and soybean oil); glycols (e.g., ethylene glycol, propylene glycol, and polyethylene glycol); polyols (e.g., glycerin, sorbitol, and mannitol); esters (e.g., ethyl oleate and ethyl laurate); agar; and other non-toxic compatible substances employed in pharmaceutical formulations. If desired, certain sweetening and/or flavoring and/or coloring agents may be added. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995). The formulations can be presented in unit-dose or multi-dose sealed containers, such as ampules and vials, and can be stored in a freeze-dried (lyophilized) condition requiring only the addition of the sterile liquid carrier, such as, for example, water, for injections, immediately prior to use. Extemporaneous injection solutions and suspensions can be prepared from sterile powders, granules, and tablets of the kind previously described.

In another aspect, the present disclosure is directed to methods of using any of the above described biocompatible radionuclide-containing compositions for treating a disease or condition in which a radionuclide (radioisotope) can be useful in treating. In the method of treating, an effective amount of the composition, typically in a pharmaceutical composition, may be administered to a human or an animal in need thereof by any of a number of well-known methods. For example, the composition may be administered systemically or locally, such as by injection. The systemic or local administration of the composition may be by intravenous, subcutaneous, intraperitoneal, intramuscular, intrathecal, or oral administration. An effective amount of a pharmaceutical composition of the invention is any amount that is effective to achieve its purpose. The effective amount, usually expressed in mg/kg can be determined by routine methods during pre-clinical and clinical trials by those of skill in the art. Dosing is dependent on the severity and responsiveness of the disease or condition being treated, with the course of treatment possibly lasting from several days to several months, or until a cure is effected or a diminution of the disease state is achieved. Optimal dosing schedules can be calculated from measurements of drug accumulation in the body of the patient. The administering physician can determine optimum dosages, dosing methodologies, and repetition rates. The dose administered to the subject should be sufficient to result in a therapeutic response in the subject over a reasonable time frame to treat an illness or condition, and/or for diagnostic imaging of the desired tissue or organ. The dose can be determined by the potency of the particular compositions employed and the condition of the subject as well as the body weight of the animal (e.g., human) to be treated. The size of the dose can also be determined by the nature and extent of any adverse side effects that may accompany the administration of a particular composition. A typical dosage for internal administration is 0.01 to 100 mg/kg of body weight per day, such as 0.01 to 35 mg/kg of body weight per day or 0.05 to 5 mg/kg of body weight per day. A typical concentration of the composition in pharmaceutical compositions for topical administration is 0.05 to 15% (by weight), preferably 0.02 to 5%, and more preferably 0.1 to 3%.

In different embodiments, depending on these and other factors, a suitable dosage of the radionuclide-containing composition may be precisely, at least, or no more than, for example, 1 mg, 10 mg, 50 mg, 100 mg, 200 mg, 300 mg, 400 mg, 500 mg, 600 mg, 700 mg, 800 mg, 900 mg, 1000 mg, 1200 mg, or 1500 mg, per 50 kg, 60 kg, or 70 kg adult, or a dosage within a range bounded by any of the foregoing exemplary dosages. Depending on these and other factors, the composition is administered in the indicated dosage or within a range thereof by any suitable schedule, e.g., once, twice, or three times a day for a total treatment time of one, two, three, four, or five days, and up to, for example, one, two, three, or four weeks or months. The indicated dosage may alternatively be administered every two or three days, or per week. Alternatively, or in addition, the composition is administered until a desired change is evidenced.

In some embodiments, the present disclosure is directed to methods of using any of the above described biocompatible radionuclide-containing compositions for treating cancer in a subject. In the method, any of the above described biocompatible radionuclide-containing compositions, typically in the form of a pharmaceutical composition, is administered to a subject having cancer in a pharmaceutically effective amount to treat the cancer. The cancer may be, for example, prostate cancer, ovarian cancer, breast cancer, leukemia, colorectal cancer, lymphoma, and neuroblastoma. The method of treating cancer may also include selectively targeting cancerous tissue by including a cancer targeting moiety, such as any of those described above, attached to the radionuclide-containing composition. The method of treating cancer may additionally (i.e., sequentially or simultaneously) include imaging of cancerous tissue that has accumulated the radionuclide-containing composition. The imaging can be achieved by any of the nuclear medicine imaging techniques known in the art.

In some embodiments, the present disclosure is directed to methods of using any of the above described biocompatible radionuclide-containing compositions for treating an infectious disease in a subject. The infectious disease may be, for example, a bacterial, viral, fungal, or parasitic infection. In some embodiments, the infectious disease is a drug-resistant infectious disease, such as methicillin-resistant Staphylococcus aureus (MRSA). The infection may also be in the form of a biofilm. In some embodiments, the method is used to treat an infected joint or prevent a joint from getting infected that is at risk of becoming infected after undergoing artificial replacement surgery or due to other conditions. In some embodiments, the carrier of the composition is attached to a targeting moiety capable of selectively transporting the composition to infected cells or joint cells when introduced into an organism. The method of treating an infection may additionally (i.e., sequentially or simultaneously) include imaging of infected tissue that has accumulated the radionuclide-containing composition. The imaging can be achieved by any of the nuclear medicine imaging techniques known in the art. Additional infections that can be imaged and or treated with the biocompatible radionuclide-containing nanoparticles described herein include tuberculosis or other granulomatous diseases, leishmaniasis or other parasitic diseases of a topical or systemic nature.

In other aspects, the present disclosure is directed to methods of imaging bodily tissue using any of the above described biocompatible radionuclide-containing compositions. The imaging method is typically for the purpose of diagnostic imaging. The imaging radionuclide may be any of those known in the art, including those described above, such as, for example, iodine-131, -124, and 123, technetium-99m, tallium-201, indium-I 11, gallium-67, cerium-134, or copper-64. In particular embodiments, a method is provided for obtaining a diagnostic image of a host by administering to the host a composition described above in an amount effective to provide an image and exposing the host to an energy source, whereupon a diagnostic image of the host is obtained. The diagnostic image can be, for example, a magnetic resonance image (MRI), fluorescence image (FI), x-ray contrast image, transmission electron microscopy image, positron emission tomography (PET) image, single photon emission computed spectroscopy (SPECT), or other type of diagnostic image.

Examples have been set forth below for the purpose of illustration and to describe certain specific embodiments of the invention. However, the scope of this invention is not to be in any way limited by the examples set forth herein.

### EXAMPLES

Synthesis of delivery platform-chelator compound radioisotope complex

A double-emulsion solvent evaporation method was adapted for the synthesis of PLGA-chelator compound radioisotope complex. The main differences include: (1) using a cup-horn sonicator to generate emulsions and (2) decreasing the reagent volumes to minimize the generation of radiological waste. In a typical synthesis, 100 μL of PLGA dissolved in EtOAc at 40 mg/mL were mixed with 10 μL of 225Ac-chelator compound radioisotope complex in a polar solution (i.e., DI H₂O, MeOH, or a mixture of both) within an Eppendorf tube. This mixture was sonicated for 30 s in a cup-horn sonicator to generate a water/oil emulsion. After the sonication was completed, 200 μL of vitamin E-TPGS (1 wt. %) in DI H₂O was added to the Eppendorf tube as an emulsifier and mixed with a vortex mixer. Three sonication cycles of 30 s with a 30 s rest time were used to generate a water/oil/water emulsion. The water in the cup-horn sonicator was kept cold by adding ice before each synthesis to prevent damage to the PLGA-chelator compound radioisotope complex. The emulsification was added to a beaker with 2.5 mL of vitamin E-TPGS (0.5 wt. %) in DI H2O or transferred into a dialysis cassette. Emulsions added to the beaker were stirred at 500-700 rpm for at least 4 hours to evaporate the organic phase and then transferred into an Eppendorf tube. Suspensions were washed at least three times by centrifugation (15,000 rpm, 20 minutes, 8° C.) and sonication in DI H₂O. The parameters and steps just mentioned are referred herein as standard conditions for the synthesis of PLGA-chelator compound radioisotope complex. Dialysis against phosphate-buffered saline (90 mL) was used to monitor radioisotope retention within PLGA-chelator compound radioisotope complex and to prepare PLGA-chelator compound radioisotope complex for cell viability experiments. Emulsions were dialyzed at least 4 hours for PLGA-chelator compound radioisotope complex synthesis, whereas for radioisotope retention studies dialysis was performed over multiple days. A schematic representation of the PLGA-chelator compound radioisotope complex synthesized with a double-emulsion solvent evaporation method using a cup-horn sonicator is shown in FIG. 1.

As described above, PLGA nanoparticles were synthesized encapsulating free ²²⁵Ac or ²²⁵Ac-BLPhen complex in a methanol/deionized water (MeOH/DI H₂O) mixture. Different MeOH/DI H₂O mixtures were prepared with 95:5, 90:10, and 75:25 MeOH:DI H₂O volume ratios to adjust the ²²⁵Ac activity. The activity of ²²⁵Ac in these solutions ranged between 0.1 μCi and 1.5 μCi (3.7 kBq and 54.8 kBq). The encapsulation efficiency of ²²⁵Ac was determined after washing the PLGA nanoparticle suspensions three times with DI H₂O. After each centrifugation, the supernatant was carefully removed with a transfer pipette and stored in a 15 mL Falcon tube. The PLGA nanoparticle precipitate was dispersed in DI H₂O using a cup-horn sonicator, and the process was repeated two more times. The PLGA nanoparticle suspension, the supernatant, and the Eppendorf tube used for sonication were assayed using γ-ray spectroscopy on a high purity germanium detector coupled to a PC-based multichannel analyzer to account for total radioactivity distribution. The HPGe has a crystal active volume of ˜100 cm³ and a beryllium window. Energy and efficiency calibrations were determined by γ-ray sources traceable to the National Institute of Standards and Technology.

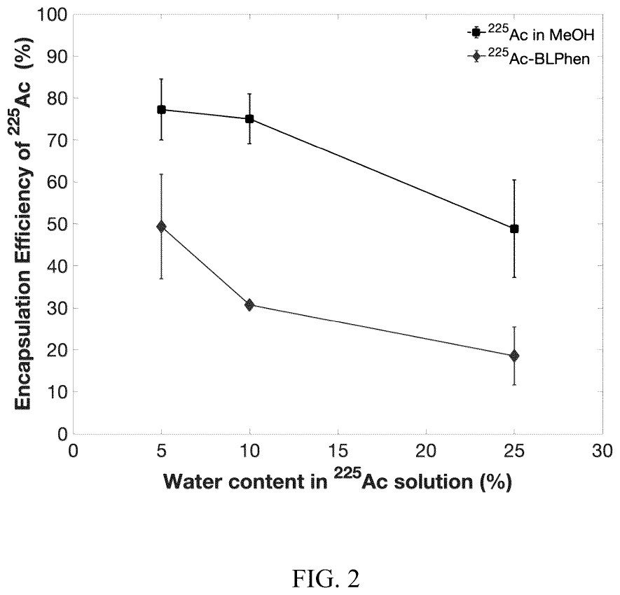

The initial PLGA encapsulation efficiency of ²²⁵Ac in a MeOH/DI H₂O mixture was higher than that of ²²⁵Ac-BLPhen for the same mixture. As shown in FIG. 2, increasing the water content in the MeOH/DI H₂O mixture decreased the encapsulation efficiency within PLGA nanoparticles for both ²²⁵Ac and ²²⁵Ac-BLPhen. The lipophilic nature of the ²²⁵Ac-BLPhen complex improved the encapsulation of ²²⁵Ac within PLGA nanoparticles by decreasing its water partitioning during nanoparticle synthesis. The encapsulation efficiency of ²²⁵Ac in a MeOH/DI H₂O mixture was studied as a control for the lipophilic BLPhen ligand. Actinium-225 in this mixture had a higher encapsulation efficiency within PLGA nanoparticles than the lipophilic BLPhen ligand for less hydrophobic environments. The lower encapsulation efficiency of ²²⁵Ac paralleled an increase in water content in the payload mixture which was associated with a decrease in miscibility with the organic phase.

Radioisotope retention was assessed by quantifying the fraction of ²²⁵Ac and ²²¹Fr activity in the dialysate over time. The water/oil/water emulsion (˜0.5 mL) was transferred into a dialysis cassette and dialyzed against phosphate buffered saline (PBS, 90 mL). Aliquots (5 mL) were taken from the dialysate at different time points (e.g., 0.5, 4, 24, 120, and 168 h) and assayed immediately on a high purity germanium detector to quantify the activity of ²²¹Fr (218 keV). A 5 mL solution of fresh PBS was added to the beaker to maintain a constant dialysate volume. The aliquot removed from the dialysate was counted the next day (decay daughters are now in equilibrium with ²²⁵A) to assess the fraction of ²²⁵Ac in the dialysate.

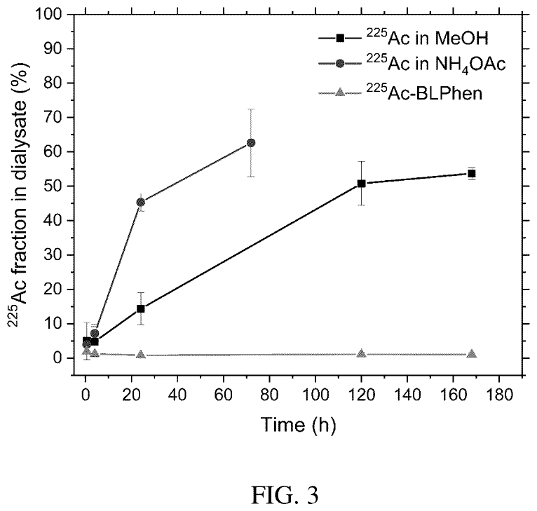

As shown by FIG. 3, retention of ²²⁵Ac within PLGA nanoparticles was influenced by the payload solution used during synthesis. PLGA nanoparticles encapsulating ²²⁵Ac in NH₄OAc displayed a substantial release of ²²⁵Ac (45.3±2.5%) after 24 h in dialysis. Using ²²⁵Ac in a MeOH/DI H₂O mixture as a payload resulted in a controlled release of ²²⁵Ac with a maximum of 53.7±1.7% of ²²⁵Ac found in the dialysate after 168 h. PLGA nanoparticles encapsulating ²²⁵Ac-BLPhen released less than 2% of ²²⁵Ac activity into the dialysate at the different time points analyzed. As also indicated by FIG. 3, retention of ²²⁵Ac within PLGA nanoparticles was influenced by its chelation to a lipophilic ligand and by the hydrophobic composition of the payload solution. A burst release of ²²⁵Ac-reaching >60% of ²²⁵Ac activity in the dialysate after 72 h was observed for PLGA nanoparticles encapsulating ²²⁵Ac reconstituted with NH₄OAc. The burst release of ²²⁵Ac may be related to a combined effect of the hydrolysis of PLGA nanoparticles, the surface adsorption of ²²⁵Ac, and the rapid diffusion of encapsulated ²²⁵Ac through the polymeric matrix into the dialysate. Encapsulation of free ²²⁵Ac in a MeOH/DI H₂O mixture resulted in a controlled release of ²²⁵Ac, with >50% in the dialysate after 168 h in dialysis (FIG. 3). Therefore, the extent of hydrophobicity within the PLGA nanoparticles (determined by the amount of water present in the ²²⁵Ac solvent) gives rise to differences in release rates as observed for ²²⁵Ac in a MeOH/DI H₂O mixture and ²²⁵Ac reconstituted in NH₄OAc (FIG. 3). The lower water content alters the rate of diffusion through water pores and the rate of PLGA hydrolysis. Encapsulation of ²²⁵Ac-BLPhen within PLGA nanoparticles permitted better retention of ²²⁵Ac within the nanoparticles with less than 2% ²²⁵Ac release over time into the dialysate (FIG. 3). The enhanced retention of ²²⁵Ac was due to the stability and lipophilic nature of the ²²⁵Ac-BLPhen complex and the low water content within these PLGA nanoparticles.

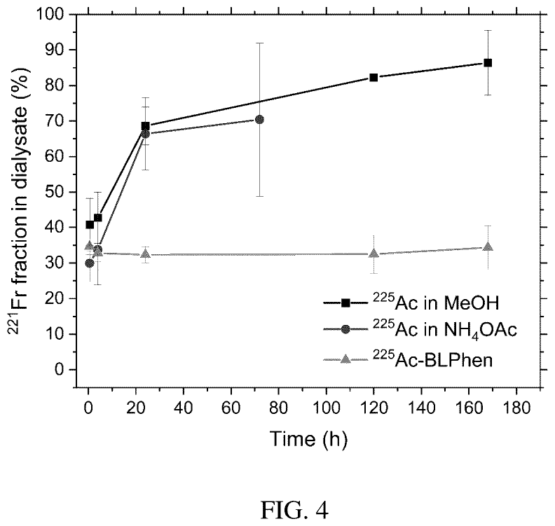

Radioisotope retention was assessed as described above. As shown by FIG. 4, release of ²²¹Fr was similar in PLGA nanoparticles encapsulating ²²⁵Ac in NH₄OAc and ²²⁵Ac in a MeOH/DI H₂O mixture, >60% after 24 h in dialysis. As also shown in FIG. 4, the fraction of ²²¹Fr released from PLGA nanoparticles encapsulating ²²⁵Ac-BLPhen varied between 32% and 35% with time. As also shown in FIG. 4, a high release of ²²¹Fr was obtained after encapsulation of ²²⁵Ac in a MeOH/DI H₂O mixture and in an NH₄OAc solution within PLGA nanoparticles. The relationship between particle size and the retention of decay daughters within polymeric nanoparticles likely contributed to these results. The adsorption of ²²⁵Ac onto the nanoparticle surface combined with an increased rate of PLGA hydrolysis can in turn increase the release of decay daughters into the dialysate. Encapsulating ²²⁵Ac-BLPhen within PLGA nanoparticles decreased the release of ²²¹Fr into the dialysate to approximately 33%. The enhanced retention of ²²¹Fr within PLGA nanoparticles can be explained by a random distribution of ²²⁵Ac-BLPhen within each nanoparticle. This random distribution of ²²⁵Ac-BLPhen within PLGA nanoparticles was likely achieved during synthesis because of the active loading.

Breast cancer cell lines (human and mouse) were exposed for 24 h to different ²²⁵Ac activities delivered as free ²²⁵Ac, ²²⁵Ac-BLPhen complex, and PLGA nanoparticles encapsulating ²²⁵Ac-BLPhen complex. These samples were prepared in phosphate buffered saline and then serially diluted into DMEM/F12 complete media. After 24 h of exposure to 225Ac, the radioactive media (200 μL/well) was removed and cells were washed three times with phosphate buffered saline. Cell viability was assessed 1 h and 72 h after exposure to ²²⁵Ac using the alamarBlue™ HS cell viability reagent. AlmmarBlue™ reagent was mixed with DMEM/F12 complete media (10 μL alamarBlue™ plus 90 μL media) and then added to the 96-well plate (100 μL per well). Cells were incubated at 37° C. for 1 h before measuring the alamarBlue™ fluorescence (λexc=560 nm and λemi=590 nm) with a BioTek Cytation 1 cell imaging multimode reader (red filter block, λexc=530 nm and λemi=590 nm). After the fluorescence was recorded, the alamarBlue™ media was removed and replaced with fresh DMEM/F12 complete media, and cells were incubated at 37° C., 5% CO₂ in the humidified incubator. The viability assay was repeated 72 h after exposure to ²²⁵Ac following the steps described previously.

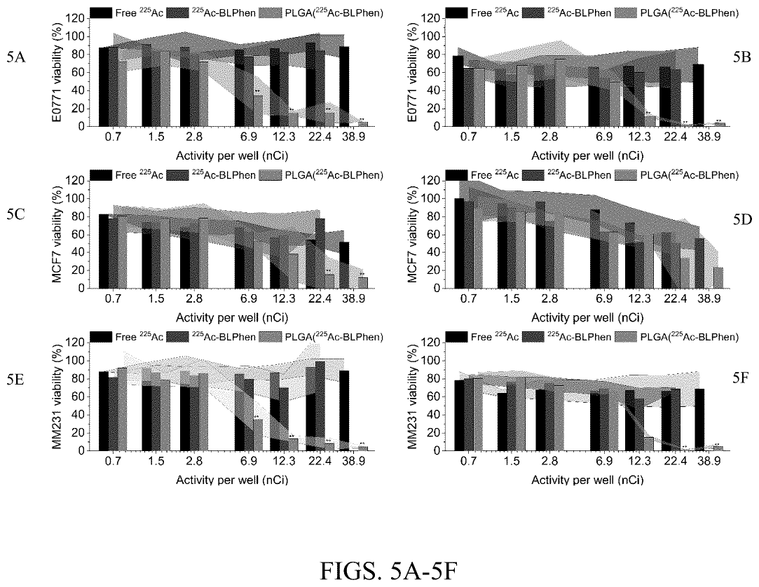

The cytotoxic effect of PLGA nanoparticles encapsulating ²²⁵Ac-BLPhen was determined using murine (E0771) and human (MCF-7 and MDA-MB-231) breast cancer cells. Free ²²⁵Ac and ²²⁵Ac-BLPhen were used as controls. FIGS. 5A-5F are bar plots showing the highly cytotoxic effect of PLGA nanoparticles encapsulating ²²⁵Ac-BLPhen complex on murine E0771 (FIGS. 5A and 5B), human MCF7 (FIGS. 5C and 5D), and MDA-MB-231 (FIGS. 5E and 5F). Free ²²⁵Ac and ²²⁵Ac-BLPhen complex were used as controls. Left-side plots (FIGS. 5A, 5C, and 5E) correspond to viability assessed 1 h post exposure to ²²⁵Ac, whereas right-side plots (FIGS. 5B, 5D, and 5F) correspond to viability assessed 72 h post exposure. Exposure of E0771 (FIGS. 5A and 5B), MCF-7 (FIGS. 5C and 5D), and MDA-MB-231 (FIGS. 5E and 5F) cells to PLGA nanoparticles encapsulating ²²⁵Ac-BLPhen significantly decreased their viability compared with exposure to controls, free ²²⁵Ac, and ²²⁵Ac-BLPhen. The spatial distribution of free ²²⁵Ac, ²²⁵Ac-BLPhen, and PLGA nanoparticles encapsulating ²²⁵Ac-BLPhen within the well and their potential uptake into cells were determining factors for the observed cytotoxic effect. Both free ²²⁵Ac and ²²⁵Ac-BLPhen will likely remain in solution uniformly distributed within the media volume in each well, decreasing the dose delivered to the cells which are attached to the bottom of the plate. Differences in viability between cell lines are likely associated with their sensitivity to α-particles and the rate of PLGA nanoparticle uptake and internalization.

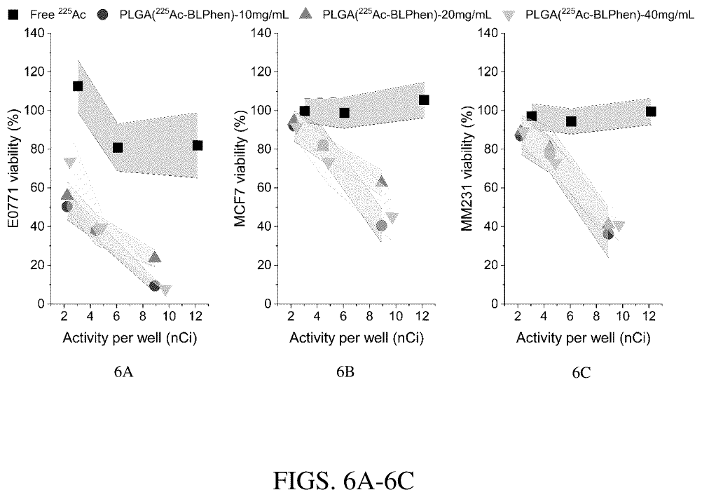

E0771, MCF-7, and MDA-MB-231 cells were exposed to different concentrations of PLGA nanoparticles (e.g., 10 mg/mL, 20 mg/mL, and 40 mg/mL) but with similar ²²⁵Ac activities to assess the effect of nanoparticle concentration on cell viability. Samples were prepared and characterized as described above. FIGS. 6A-6C are graphs showing the cytotoxic effect of PLGA nanoparticles encapsulating ²²⁵Ac-BLPhen complex based on particle concentration. Viability of murine E0771 (FIG. 6A), human MCF7 (FIG. 6B), and MDA-MB-231 (FIG. 6C) was assessed 1 h post exposure to ²²⁵Ac. Delivering similar ²²⁵Ac activities with different concentrations of PLGA nanoparticles encapsulating Ac-BLPhen complex (e.g., 10 mg/mL, 20 mg/mL, and 40 mg/mL) did not significantly affect the viability of E0771 (FIG. 6A), MCF-7 (FIG. 6B) and MDA-MB-2 (FIG. 6C). As shown in FIGS. 6A-6C, the viability of cells exposed to PLGA nanoparticles encapsulating ²²⁵Ac-BLPhen was different from that of free ²²⁵Ac when using ²²⁵Ac activities greater than 4 nCi/well for all cell lines. The concentration of PLGA nanoparticles in solution did not induce cytotoxic effects in the human and murine breast cancer cells, which confirms that PLGA is a biocompatible delivery vehicle. The relative fraction of ²²⁵Ac-BLPhen within PLGA nanoparticles likely determines the cytotoxic effect since cell internalization of PLGA nanoparticles at low and high concentrations was not significantly different.

While there have been shown and described what are at present considered the preferred embodiments of the invention, those skilled in the art may make various changes and modifications which remain within the scope of the invention defined by the appended claims.

What is claimed is:

1. A biocompatible radionuclide-containing composition comprising:
(i) a radionuclide-containing core; and
(ii) a carrier encapsulating the radionuclide-containing core.

2. The composition of claim 1, wherein the radionuclide-containing core has a diameter of less than 1 micron.

3. The composition of claim 1, wherein the radionuclide-containing core has a diameter within a range of 50-900 nm.

4. The composition of claim 1, wherein the radionuclide-containing core comprises a radionuclide-chelator complex.

5. The composition of claim 4, wherein the chelator in the radionuclide-chelator complex is a phenanthroline, dipicolinate, or nitrogen-containing macrocycle compound.

6. The composition of claim 4, wherein the chelator in the radionuclide-chelator complex is a phenanthroline compound.

7. The composition of claim 1, wherein the radionuclide-containing core comprises a metal-organic framework containing radionuclide atoms coordinated with organic molecules in an extended framework.

8. The composition of claim 1, wherein the carrier is a polyester.

9. The composition of claim 8, wherein the polyester is selected from the group consisting of polylactic acid (PLA), polyglycolic acid (PGA), polycaprolactone (PCL), poly(alkyl cyanoacrylates), poly(ortho esters), polyanhydrides, and polyphosphoesters, and copolymers and mixtures thereof.

10. The composition of claim 1, wherein the carrier is a micelle or liposome.

11. The composition of claim 1, wherein the radionuclide is an alpha emitter.

12. The composition of claim 11, wherein the alpha emitter is selected from the group consisting of actinium-225, thorium-227, thorium-226, radium-223, radium-224, lead-212, astatine-211, uranium-230, bismuth-231, and combinations thereof.

13. The composition of claim 1, wherein the radionuclide is a beta emitter.

14. The composition of claim 13, wherein the beta emitter is selected from the group consisting of lutetium-177, samarium-153, yttrium-90, strontium-89, and iodine-131.

15. The composition of claim 1, wherein the carrier is attached to a targeting moiety capable of selectively transporting the composition to a specific cell type when introduced into an organism.

16. The composition of claim 1, wherein the carrier is attached to a moiety that evades an immune system of an organism.

17. A method for treating cancer in a subject, the method comprising administering to said subject a pharmaceutically effective amount of a biocompatible radionuclide-containing composition comprising:
(i) a radionuclide-containing core; and
(ii) a carrier encapsulating the radionuclide-containing core.

18. The method of claim 17, wherein the radionuclide-containing core comprises a radionuclide-chelator complex.

19. The method of claim 17, wherein said cancer is selected from the group consisting of prostate cancer, ovarian cancer, breast cancer, leukemia, colorectal cancer, lymphoma, and neuroblastoma.

20. A method of treating an infection in a subject, the method comprising administering to said subject a pharmaceutically effective amount of a biocompatible radionuclide-containing composition comprising:
(i) a radionuclide-containing core; and
(ii) a carrier encapsulating the radionuclide-containing core.

21. The method of claim 20, wherein the infection is a bacterial or fungal infection.

22. The method of claim 20, wherein the radionuclide-containing core comprises a radionuclide-chelator complex.

23. A method of imaging biological tissue in a subject, the method comprising administering to said subject a pharmaceutically effective amount of a biocompatible radionuclide-containing composition comprising:
(i) a radionuclide-containing core; and
(ii) a carrier encapsulating the radionuclide-containing core; and
imaging biological tissue in the subject by a nuclear medicine imaging technique.

24. The method of claim 23, wherein the radionuclide-containing core comprises a radionuclide-chelator complex.
